SEQUENCE LISTING (27 sequences)

SEQ ID NO: 1 - DNA, 9921 bp, Oryza officinalis
  gene (1)..(9921)
cccgggcctttctccagtatctccatgatgattgtttggattaacagacctccctggagatttacccttgcgggcattttcgaaatactgtgtgtagaggtgttcaccacggtcttcatcctcccagcttccaaatttaggaacttgtttatgctggtagagattaacaaacaagtaccatttgcgttaggcagacagcatatgctgattgctgaatctactacacgggcttgttgccttgtcagttgtcatctttcatatgtaacaggaaatagtatataattctagcctaaacaggtattccatccgtatatttctagattcattagcatcaatataaatatcggaaatactagaatgacttacattgtaaaacagagaaagtatgattatctaaactctcagatagtctattgtaatcattgtatgcatgacatcagcttgtgtcaaagctgcagctttgttggatcaaccagaatttactagttctaaaaaaaacagaatttacttagatcctgctgaaagacttcgggccaattagaagcccagtatttggcccaaaactggtgtaaactacaggcccataatagaaataagttcactggaggtcccccaacttaacaccgagatttttttgaggtcccttaaccataaaccagaaatgcgtacccctaaactatgtaaaaccgtcaaaaaaaaaaaggtcccgaggcagtatagttgctcggtttcgctgacgtggcatcctagtcagcaaaaaaaattaaaataaatatgtggggcccacatgtaagtgagagaaaatggtgtgggccccacatttctcctttcttttctttttctcttctcttctctcccgattttctcgagtgggcgcggcggcagaggggagagaggtgaccggcggtggagcgggggtggtgggagaggggggctggcgcgacgtcggcggcgggagaaaaggagaggaggcggctggcgcgacggcgagctcggacggcaacggcgggaggaggaggaggagaggaggcagtcggcgctgtaaaacgcgcatgggatgcggcggatgatgtcgagcgggacggtgtcctcatcaccatcttcctcatcgtcatcgcagtctgcgggtgccgggcggagcggtttgggagggagggggaggcggccagcgagcccgttcgagctcgcccggcctgcgcgcgagctcgcccctcccatagcttggtgtgctggggacgacgacgaggaaagggggagaaaggaattgacggccgatgatagcaatggcgggcgagcgcggcgccgggctgctcatgctcgccgaccaggcccccggcttcgccgcactcgtccgccgtgctcgcccacgcactcgccgacctccgctccacccgccgctcctgcccgctgcccgctgcccgccggcctccctgactgaagaagagaagagagaaattaaagagaagagaaggagaagaaaagagaagagaagaaaaaaagatgtgtagctgacatgtgggccccatgtacttttttattttttttgctgactagaatgccacgtcagcgaaaccacccatatatactgtcataggaccttgagtgcatagtttgtgtgagtttagggatacacatttctcgttttgtagttaagggacctcgtaaaaatctcgctgttaagttgagagacccctccggtgaacttattcccccaaatattttctccatgctctgggctcaaacgcactcttggatgggccgtgtaatctacaccatttctggcccaatccaacgaaggtagctccagactcctaactccgccgccgcatgcccaatcctcgccggccgccgctgccatctcccttcccccaccgccaccgtcgccttctccgttcccccaacaaaggctcagtagcagcagccgcggcgcggagagcagaggccttggagggcctgcagaggagcgcccgcgtcgacgaagacgacatcagcaggcagtggacatggcgactgcgaatgctatcgccgacgtgggagcttcaggcgaggatgcgtggccttcatcagctgctgctcccttacctgctctggtttgtcattgtgttggttcgtggtttggggatatcaggatggggaaggggaatgggtttcgtgccatgttgttctattgtgcaatttgatgttctcttgtcgaatttcatggtgattcaatctgcatctgtgttattggttgtcaagcaaacattttatttctttcgagatgggtgtttcatctgaaattggtgtgtctgatgatgtgaccttgtgcaaatcattgtttcattttcttctattccacagagatttttttgtttagagtttattctaccatcttcttaccttttgaatgctatgcaccacagatttctgatttttacacattcaatcacagtgccgacccttggcgaatttaaaattttctcaccgtcaaaatttaatcaaacatctgatctaacagtagtagcgccagcacggctggtatttaaggcagaaagaaaggttgcgagggcgaggagttggtgatgatctgccggtcgtgtcgtgactttaggctataaaagatagctgtttggattaacccaaccaatgaagcgaatcatcgatggccgatagcgagattatatccaactaatgaagagaatcaactttaatcttttgtgagctacaagtttcgtggtgaaggcatatactaataccaccatcatggctgcagcgaatcaacgagatgcatttgtcttgcacatttcccaccggatactttgccatccatggctttatctcctccatgattgtatccccttcttcctcaacaaggaacgatcgatcattgtttgcttctttctctgacgacactttggtgcaagtgaaaggctctttcctggaactcgtcccgtgagctccttcctggctacatcctccttaattccttgccgatttccggcttcgtcttcactatcgtcagttcatttctcccgctctgcctgagagtccagtttcttggagatagtcgccggcttgctctctctctctctctcccttctcatcgccagctccagctcacaatccgagcttacgtggtggaatcgctatcgccacctctcggaagattattgtactttgccttgcctacttcttgagtccgatcaaacacatccatcatgcatctgtttgtacttgcattcagccaaacacttccagtttaactccgtccgcaagtcaatacttgcattcaatttccatttcctacaaaagcagctgctcagactcttgccttctccacccagcaatcgtgtttccttccctgtatcctgcgcgtgtgttcttagctgctctaggggatccttccaatggcggagctaatggccaccatggtggtcgggccactgctgtccatggtgaaggacaaggcctccagctacctcctggagcagtacaaggtgatggagggcatggaggagcagcacgagatcctcaaacgcaagctgccagccatcctcgacgtcatcgccgacgccgaggagcaggcggctaaacacagggaaggggtgaaagcatggctcgaggcgctccggaaggtggcctaccaggccaatgacgtcttcgacgagttcaagtacgaggcactccgccgcaaggccaaggggcactacaagatgctcagcagcatggttgtaatcaagctcattcctactcacaaccgtattctgttcagttataggatgggcaacaagctcaggatgattctgaatgccattgaagttctaattgaagagatgaatgcctttaggtttaaattccgaccagagccaccaatgtcgtccatgaaatggaggaagacagattctaaaatctccgacctttcaaagctaagtgacttagagatatctaaaggcaatcaacaaatatccctacaggcagccagcagacatattacttcattgtccagtctcgttctgcatttgtccactgatgacacagaaacagcatcggtggccaagcaacaagattcgagtgatttggtgattgaggatgagaaatggagtcataaatctcccctggaacttatggtcttgagtcggtgcaaccttttattctctcacccaagtgcactggctctgtggacatgttttgctcagctcctagatctgaaaattcggtatgttgatgcgcttgtcagctggccagaagaggtgttccagggcttagtttccttgaggaagttagagatttctgtatgcgagaatctgacaggacacacacaagctcgtgggcaatctacacccgcaccaagtgaactcctgccacgtttggagtccctagagataacgtgttgtgattctattgtggaggtccccaatctaccggcgtctctcaagctattagaaattagggggtgccccggcctggagtccatcgtattcaatcagcagcaggataggacgatgttggtgagtgcagaaagctttgcagagcaggataagtcatcgttaatatcagggtccacaagcgagaccaacgatcacgtccttccacgcctagaatctcttgtaataaattggtgcgatcgtttggaggttctccatcttcctccgtccatcaagaaattgggtatttatagctgtgaaaaacttcggtccctctcagtaaagctggatgccgttcgagaattaagtatcagacattgcgggagcttgaaatcactggaatcttgcttaggagagctcgcgtcgctgcaacaactcaaactttttgattgcaagagcctggaatccttgccgaaggggcctcaagcatactcatctcttacatctcttgaaattcgtggttgttctggtataaaggtgcttccaccgagcctacagcaacgtctggatgacatcgaggacaaagaactagatgcctgctatgaaggtaatcttcagtttcttaaccgtgtaccatttagtggtaaaagtttcgagtttcgtgtctagaacccctagtcaaccattaatatgattatatgtacatagagtacaatgcgcattcataactcacttctgcagctgtgtcatctaaaccctttaaactttgagttgcatttgggtatctaatcgcatgcaaaggaatttagttatatctcccgtagccattccttatatgtgatgatctcttcctgtgattatgcttgctagtttggactatgtaattaattttgccgggtgactatgtaattacatgacttcatttagtcgccaggtgtggcatcatgcaattattatggcaaagctgtattagtcatgatcgaagccacttggtgaacttattccctgctactttgaaccaatactcattgattatttccctttaagcgtttgatatggacgacagtttaaatttgcagagctaactaacgcagcgcttgtctttacatttctctggaaactcacagctccattgcatatgtagtttgagtatacatagtttctttgaactgtcttatgcctttgaaaagcttgactatggaattctagtctatatattcaatacatatatttttttttcttttgtttgccagaggcagaagcagaaccaaagtctcgtcatcgtcaatctgcaatcagtaggctgatgtgcttgaagtagcagtttcaggaccagatgagagttgtcatcatggtttgtaacgcgtgcttgatgattgatttcgaatgaaattctagccactatccttatgcttactctctctatggtgtgacttccttgtaggatcgttaccgaatctgcaattgttgggttattaattgtgtggttgtatctcatggtagaatgtattacctacctaccctttctcaagataaactccagtaaagtcgatcgtctggttggtgtgagctgctacaagaaggatgaagccgatgatcccaaggccttgactttaagctagtcccaggttgtaagctgaagaacaacatactggagcttaaactgcttcactctttttttatccttttccttttcttgtgaactgtgatgtgtttgatctcgttcatgctccatcataagagaatcacagctagcacttccatttcaatgatgtttttcctaacatgacatgactgcagaggatacttgtgattgatcaacaacattttgcagcaacgttctctcccgataacgctgcaatggaattgatcaattggtgttcctacttattggaatctttggacattgccaacaactcaagaaaggaagataaacaggagattgtcagcagattgcttgttccagccagcgaaggggatctcactgttcttcccattgtaggaatgggggggatgggcaagaccaccttagcgcagctcatttacaatgaccctgacattcagaagcatttccagttgctgctctgggtgtgtgtttccgacaacttcgatgtggatttgctggctaaaagcatagttgaagcagctcgcaaacagaagaatgataacagtggaagtactaacaagtcaccattggatgaacttaaagaagttgtgagtgggcagaggtacctcctcgttttggatgatgtctggaaccgtgatgctcgtaagtgggaagcgctcaagtcctaccttcagcacggtggcagcggtagctcagttttgacaacaactcgtgatcaagaagtggctcaagtgatggctccagctcaaaaaccttatgatctcaagagactgaaggaaagcttcatagaggaaattatcaggacaagtgctttcagttcacaacaagaaaggcctcctgagcttctcaaaatggttggtgatattgccaagaaatgttctggttcccctttagctgcaacagcattgggctctacactgcgtacgaagaccaccaagaaagaatgggaggctatattaagcagaagcacaatttgcgatgaggaaaatggaattttaccaatactcaagctcagttacaattgcttgccatcatatatgcggcaatgcttttccttttgtgcaattttccccaaggatcatgagattgacgtggaaatgctgatccagttatggatggccaatggttttatcccagagcaacaaggagagtgccctgaaatcattggtaaaagaattttcagtgagttggtgtcaaggtcattttttcaggatgcgaaagggatcccgtttgagttccatgatataaagaactctaagattacttgtaagatccatgaccttatgcatgatgttgcacaatcctccatgggaaaagaatgcgctgctatagatacagaagttagtaaaagtgaggattttccttattctgctcgccatctatttttgtcaggtgatagaccagaagctattcggactccttccccagagaaaggatatccaggtatccaaacattaatatgttcacgtttcaaatatttgcagaatgtatcaaaatacaggtcattgcgagtattaacaacgatgtgggaaggttcattcctgataccaaaatatcatcatcacctgaggtatcttgatctctcagaaagtgaaattaaagcacttcctgaagacataagcatcctatatcatttgcaaacattgaacctttcccgttgtttatctctccgtcgacttccaaagggaatgaagtacatgaccgccctccgtcacttgtacactcacggatgttggagtttaggaagcatgcctcctgacctcggacacctcacttgcctacagacgcttacatgctttgtagccggtacttgctctggctgcagtgatttgggagagctgcggcagttggaccttggtggtcgactagagctaagaaaactggaaaatgtgacaaaagctgatgcaaaagcagcaaatctcggaaagaaggaaaaactgaccaaattgaccttaatatggactgatcaggagtacaaggaggcacagagtaataatcataaagaggtgctggaaggtctcacgcctcacgaggggctcaaggttctgagtatatatcactgtgggagcagtacatgtccaacttggatgaataaactgcgggacatggtggggcttgagttaaatggttgcaaaaatctcgagaagcttcctccgttgtggcagctaccggctctacaagttctttgcctggaaggactgggtagtttaaattgcttgttcaactgtgacacacacacacccttcacattttgcagactgaaggagctaaccttgtctgatatgacaaattttgagacatggtgggacacaaatgaggtacaaggagaagagctgatgtttcctgaggttgaaaagctgtcaatcgaaagttgccataggctaactgccttgccaaaagcatcaaatgcgatttcagaatcgtccggcgaagttagcaccgtgtgtcgttctgcatttccagcattgaaggaaatgaaattatatgatttgcgtatctttcagaaatgggaggcagtcgatggaactccaagggaggaggcaacatttcctcagcttgacaaattagaaatcagacagtgcccagagctgactactctacctgaagcacgaatttcccattgtggaataaaatggaaagattggtacaatctgctgaaaaatgaactgtcgttactcaagttgaaacatgtgtcatgtgtgtaactatcagaattgttctcgacaaaatcgttgggacttgcaaggtaaaaaaaaaaaattcctcagctctgtaaatgctattgctgaactaaagttcagaaagctatagtgccgaaagatctcagatggcctactgcaacaaaattcagtacaaatgcaaaacaatgtaacattacgtggcccagcccatcatggtaagcccatactgtatgcgctgcaaactaatattagttcaggcaattactctgaaatttctgatagacgaacctgattttttcatgtcaggtgccgtgcttgttcagattcagcaacttacatttccaaagaagaaaaaggaaattcataccttctctttatttggtttttaaaagtggcctggattttcattatttggttaaaatttggcctttgtcgtaaggatttggatcctctatccaaacacatgccttccaagataaacgaatccctcattatacaaaaaaaaaagcacaagatgctcaacaagaagcttcaaattcgtgtaaatacatgtcatgtctcgctaccaaacaatatctcaacaatgacaacagattggaggtattcatctcacaatgatacatttcaacacaaatttatgtaggcctcgctgacaggacttggctggatggctgtcagttcagttcttgaaccaactgaagcatgagcagccctgcaaggaagcgataaactgaattaaaataggaatttgttacacaatacctgtaaagtggattacattaaaatattaccaaatcatactgtaagcaatcatacagtacaagattccttgcaaacaaccctaaccaaacttctagtaaaaccattattttttctgtagtaccaactttttgcaaccactatatactccagtaaaaaccagaagtattgttgcaatgactttgccgataaattatagaacgcggctactcgaagcagtagatttaagacaatttttactgcataataactacgcatggtgtcaagatccctaaaaattaacagaggcagacccagctcaagtattgacaaactcaagtggcaactggcatatgaacaattttctcgttcaaatatatctgaagcaatgtgtaaaacaaattagttttgctccacacatctgacaataatttttttcagtaagcagacttactgtggattcatatttgcgttgttgatagcgtttgatataatctgcctcgctagtcatgacgggagcgttgccggtacc

SEQ ID NO: 2 - DNA, 3972 bp, Oryza officinalis
  CDS (1)..(3972)
atggcggagctaatggccaccatggtggtcgggccactgctgtccatggtgaaggacaaggcctccagctacctcctggagcagtacaaggtgatggagggcatggaggagcagcacgagatcctcaaacgcaagctgccagccatcctcgacgtcatcgccgacgccgaggagcaggcggctaaacacagggaaggggtgaaagcatggctcgaggcgctccggaaggtggcctaccaggccaatgacgtcttcgacgagttcaagtacgaggcactccgccgcaaggccaaggggcactacaagatgctcagcagcatggttgtaatcaagctcattcctactcacaaccgtattctgttcagttataggatgggcaacaagctcaggatgattctgaatgccattgaagttctaattgaagagatgaatgcctttaggtttaaattccgaccagagccaccaatgtcgtccatgaaatggaggaagacagattctaaaatctccgacctttctttggacattgccaacaactcaagaaaggaagataaacaggagattgtcagcagattgcttgttccagccagcgaaggggatctcactgttcttcccattgtaggaatgggggggatgggcaagaccaccttagcgcagctcatttacaatgaccctgacattcagaagcatttccagttgctgctctgggtgtgtgtttccgacaacttcgatgtggatttgctggctaaaagcatagttgaagcagctcgcaaacagaagaatgataacagtggaagtactaacaagtcaccattggatgaacttaaagaagttgtgagtgggcagaggtacctcctcgttttggatgatgtctggaaccgtgatgctcgtaagtgggaagcgctcaagtcctaccttcagcacggtggcagcggtagctcagttttgacaacaactcgtgatcaagaagtggctcaagtgatggctccagctcaaaaaccttatgatctcaagagactgaaggaaagcttcatagaggaaattatcaggacaagtgctttcagttcacaacaagaaaggcctcctgagcttctcaaaatggttggtgatattgccaagaaatgttctggttcccctttagctgcaacagcattgggctctacactgcgtacgaagaccaccaagaaagaatgggaggctatattaagcagaagcacaatttgcgatgaggaaaatggaattttaccaatactcaagctcagttacaattgcttgccatcatatatgcggcaatgcttttccttttgtgcaattttccccaaggatcatgagattgacgtggaaatgctgatccagttatggatggccaatggttttatcccagagcaacaaggagagtgccctgaaatcattggtaaaagaattttcagtgagttggtgtcaaggtcattttttcaggatgcgaaagggatcccgtttgagttccatgatataaagaactctaagattacttgtaagatccatgaccttatgcatgatgttgcacaatcctccatgggaaaagaatgcgctgctatagatacagaagttagtaaaagtgaggattttccttattctgctcgccatctatttttgtcaggtgatagaccagaagctattcggactccttccccagagaaaggatatccaggtatccaaacattaatatgttcacgtttcaaatatttgcagaatgtatcaaaatacaggtcattgcgagtattaacaacgatgtgggaaggttcattcctgataccaaaatatcatcatcacctgaggtatcttgatctctcagaaagtgaaattaaagcacttcctgaagacataagcatcctatatcatttgcaaacattgaacctttcccgttgtttatctctccgtcgacttccaaagggaatgaagtacatgaccgccctccgtcacttgtacactcacggatgttggagtttaggaagcatgcctcctgacctcggacacctcacttgcctacagacgcttacatgctttgtagccggtacttgctctggctgcagtgatttgggagagctgcggcagttggaccttggtggtcgactagagctaagaaaactggaaaatgtgacaaaagctgatgcaaaagcagcaaatctcggaaagaaggaaaaactgaccaaattgaccttaatatggactgatcaggagtacaaggaggcacagagtaataatcataaagaggtgctggaaggtctcacgcctcacgaggggctcaaggttctgagtatatatcactgtgggagcagtacatgtccaacttggatgaataaactgcgggacatggtggggcttgagttaaatggttgcaaaaatctcgagaagcttcctccgttgtggcagctaccggctctacaagttctttgcctggaaggactgggtagtttaaattgcttgttcaactgtgacacacacacacccttcacattttgcagactgaaggagctaaccttgtctgatatgacaaattttgagacatggtgggacacaaatgaggtacaaggagaagagctgatgtttcctgaggttgaaaagctgtcaatcgaaagttgccataggctaactgccttgccaaaagcatcaaatgcgatttcagaatcgtccggcgaagttagcaccgtgtgtcgttctgcatttccagcattgaaggaaatgaaattatatgatttgcgtatctttcagaaatgggaggcagtcgatggaactccaagggaggaggcaacatttcctcagcttgacaaattagaaatcagacagtgcccagagctgactactctacctgaagcaccaaagctaagtgacttagagatatctaaaggcaatcaacaaatatccctacaggcagccagcagacatattacttcattgtccagtctcgttctgcatttgtccactgatgacacagaaacagcatcggtggccaagcaacaagattcgagtgatttggtgattgaggatgagaaatggagtcataaatctcccctggaacttatggtcttgagtcggtgcaaccttttattctctcacccaagtgcactggctctgtggacatgttttgctcagctcctagatctgaaaattcggtatgttgatgcgcttgtcagctggccagaagaggtgttccagggcttagtttccttgaggaagttagagatttctgtatgcgagaatctgacaggacacacacaagctcgtgggcaatctacacccgcaccaagtgaactcctgccacgtttggagtccctagagataacgtgttgtgattctattgtggaggtccccaatctaccggcgtctctcaagctattagaaattagggggtgccccggcctggagtccatcgtattcaatcagcagcaggataggacgatgttggtgagtgcagaaagctttgcagagcaggataagtcatcgttaatatcagggtccacaagcgagaccaacgatcacgtccttccacgcctagaatctcttgtaataaattggtgcgatcgtttggaggttctccatcttcctccgtccatcaagaaattgggtatttatagctgtgaaaaacttcggtccctctcagtaaagctggatgccgttcgagaattaagtatcagacattgcgggagcttgaaatcactggaatcttgcttaggagagctcgcgtcgctgcaacaactcaaactttttgattgcaagagcctggaatccttgccgaaggggcctcaagcatactcatctcttacatctcttgaaattcgtggttgttctggtataaaggtgcttccaccgagcctacagcaacgtctggatgacatcgaggacaaagaactagatgcctgctatgaagaggcagaagcagaaccaaagtctcgtcatcgtcaatctgcaatcagtaggctgatgtgcttgaagtag

SEQ ID NO: 3 - protein, 1323 aa, Oryza officinalis
MAELMATMVVGPLLSMVKDKASSYLLEQYKVMEGMEEQHEILKRKLPAILDVIADAEEQAAKHREGVKAWLEALRKVAYQANDVFDEFKYEALRRKAKGHYKMLSSMVVIKLIPTHNRILFSYRMGNKLRMILNAIEVLIEEMNAFRFKFRPEPPMSSMKWRKTDSKISDLSLDIANNSRKEDKQEIVSRLLVPASEGDLTVLPIVGMGGMGKTTLAQLIYNDPDIQKHFQLLLWVCVSDNFDVDLLAKSIVEAARKQKNDNSGSTNKSPLDELKEVVSGQRYLLVLDDVWNRDARKWEALKSYLQHGGSGSSVLTTTRDQEVAQVMAPAQKPYDLKRLKESFIEEIIRTSAFSSQQERPPELLKMVGDIAKKCSGSPLAATALGSTLRTKTTKKEWEAILSRSTICDEENGILPILKLSYNCLPSYMRQCFSFCAIFPKDHEIDVEMLIQLWMANGFIPEQQGECPEIIGKRIFSELVSRSFFQDAKGIPFEFHDIKNSKITCKIHDLMHDVAQSSMGKECAAIDTEVSKSEDFPYSARHLFLSGDRPEAIRTPSPEKGYPGIQTLICSRFKYLQNVSKYRSLRVLTTMWEGSFLIPKYHHHLRYLDLSESEIKALPEDISILYHLQTLNLSRCLSLRRLPKGMKYMTALRHLYTHGCWSLGSMPPDLGHLTCLQTLTCFVAGTCSGCSDLGELRQLDLGGRLELRKLENVTKADAKAANLGKKEKLTKLTLIWTDQEYKEAQSNNHKEVLEGLTPHEGLKVLSIYHCGSSTCPTWMNKLRDMVGLELNGCKNLEKLPPLWQLPALQVLCLEGLGSLNCLFNCDTHTPFTFCRLKELTLSDMTNFETWWDTNEVQGEELMFPEVEKLSIESCHRLTALPKASNAISESSGEVSTVCRSAFPALKEMKLYDLRIFQKWEAVDGTPREEATFPQLDKLEIRQCPELTTLPEAPKLSDLEISKGNQQISLQAASRHITSLSSLVLHLSTDDTETASVAKQQDSSDLVIEDEKWSHKSPLELMVLSRCNLLFSHPSALALWTCFAQLLDLKIRYVDALVSWPEEVFQGLVSLRKLEISVCENLTGHTQARGQSTPAPSELLPRLESLEITCCDSIVEVPNLPASLKLLEIRGCPGLESIVFNQQQDRTMLVSAESFAEQDKSSLISGSTSETNDHVLPRLESLVINWCDRLEVLHLPPSIKKLGIYSCEKLRSLSVKLDAVRELSIRHCGSLKSLESCLGELASLQQLKLFDCKSLESLPKGPQAYSSLTSLEIRGCSGIKVLPPSLQQRLDDIEDKELDACYEEAEAEPKSRHRQSAISRLMCLK

SEQ ID NO: 4 - DNA, 24 bp, Artificial Sequence
  primer
ctccctgactgaagaagagaagag

SEQ ID NO: 5 - DNA, 25 bp, Artificial Sequence
  primer
tgctagctgtgattctcttatgatg

SEQ ID NO: 6 - DNA, 32 bp, Artificial Sequence
  primer
cggaattcctccctgactgaagaagagaagag

SEQ ID NO: 7 - DNA, 33 bp, Artificial Sequence
  primer
cggaattctgctagctgtgattctcttatgatg

SEQ ID NO: 8 - DNA, 30 bp, Artificial Sequence
  primer
tccccccgggatggcggagctaatggccac

SEQ ID NO: 9 - DNA, 30 bp, Artificial Sequence
  primer
gctctagactacttcaagcacatcagccta

SEQ ID NO: 10 - DNA, 28 bp, Artificial Sequence
  primer
cggaattcatggtggagcacgacactct

SEQ ID NO: 11 - DNA, 30 bp, Artificial Sequence
  primer
tccccccgggatctcattgccccccgggat

SEQ ID NO: 12 - DNA, 20 bp, Artificial Sequence
  primer
ctgctgctgctctcgtattg

SEQ ID NO: 13 - DNA, 20 bp, Artificial Sequence
  primer
cagggaagctccaagaacag

SEQ ID NO: 14 - DNA, 20 bp, Artificial Sequence
  primer
tccacctttgcaaatccaat

SEQ ID NO: 15 - DNA, 20 bp, Artificial Sequence
  primer
cttcttcctctggctggcta

SEQ ID NO: 16 - DNA, 20 bp, Artificial Sequence
  primer
ccggcgagaagttctacctc

SEQ ID NO: 17 - DNA, 20 bp, Artificial Sequence
  primer
gctgtaatcctgcgtgtcct

SEQ ID NO: 18 - DNA, 21 bp, Artificial Sequence
  primer
tgtgggttttgcttctcactt

SEQ ID NO: 19 - DNA, 22 bp, Artificial Sequence
  primer
caaaccactgtgagtacggcta

SEQ ID NO: 20 - DNA, 26 bp, Artificial Sequence
  primer
agaaatggtgaaagatggtgctaccg

SEQ ID NO: 21 - DNA, 25 bp, Artificial Sequence
  primer
ctgaatgttcttcaactcccagtgc

SEQ ID NO: 22 - DNA, 20 bp, Artificial Sequence
  primer
agcgttaagcgccattatca

SEQ ID NO: 23 - DNA, 20 bp, Artificial Sequence
  primer
cgccgaggcattagagtaga

SEQ ID NO: 24 - DNA, 20 bp, Artificial Sequence
  primer
ctgctgctgctctcgtattg

SEQ ID NO: 25 - DNA, 20 bp, Artificial Sequence
  primer
cagggaagctccaagaacag

SEQ ID NO: 26 - DNA, 21 bp, Artificial Sequence
  primer
gcaaatatgtgcatacctgga

SEQ ID NO: 27 - DNA, 18 bp, Artificial Sequence
  primer
ctgacatctccgcctgga

(19) United States
(12) Patent Application Publication
(10) Pub. No.: US 2011/0287966 A1
(43) Pub. Date: Nov. 24, 2011

(54) MOLECULAR MARKERS FOR RICE BROWN PLANTHOPPER RESISTANCE GENE

(75) Inventors: Guangcun He, Hubei (CN); Bo Du, Hubei (CN); Weilin Zhang, Hubei (CN); Lili Zhu, Hubei (CN); Rongzhi Chen, Hubei (CN)

(73) Assignee: WUHAN UNIVERSITY, Hubei (CN)

(21) Appl. No.: 13/143,454

(22) PCT Filed: Dec. 30, 2009
(86) PCT No.: PCT/CN09/76244

§ 371 Date: Aug. 12, 2011

(51) Int. Cl. C40B 30/04; C07H 21/04; C12Q 1/68

(52) U.S. Cl. 506/9; 435/6.12; 536/23.1

(57) ABSTRACT

Molecular markers for rice planthopper resistance gene Bph14 are provided. The Bph14 gene belongs to the CC-NBS-LRR gene family, and its coded protein is related to plant disease resistance. Bph14 gene has the function of resisting brown planthopper. By determining Bph14 gene molecular markers in rice varieties, breeding is improved. Harm caused by brown planthopper can be alleviated and the aim of increasing and stabilizing production can be achieved.

### FIELD OF THE INVENTION

The present invention belongs to the field of plant gene engineering. Specifically, it relates to a rice brown planthopper resistance gene Bph14 and the use of the gene in rice and rice seed to resist brown planthopper.

### BACKGROUND OF THE INVENTION

Rice is a very important food crop, which serves as staple food for more than half of the world's population. Furthermore, detailed genetic mapping and physical mapping of the rice genome is complete. Creating transgenic rice has become routine. Additionally, rice has colinearity with the genomes of other gramineous crops, therefore it has been viewed as a model plant. Therefore, the study of rice functional genes has significant meanings for social economic development and biological research.

The lack of adequate food supply is a challenge faced by the entire world. Rice yield has been dramatically increased by the two technology revolutions of dwarf rice plant of the 1950s and 1960s, and the hybrid rice of the 1970s. However, rice crops are still harmed by pests over large areas and rice production remains threatened, particularly by brown planthopper. Brown planthopper adults and nympha stab and suck the rice sap with their stylets, causing the leaves to turn yellow or to wither to death, which results in reduction or total destruction of the yield. According to China agriculture yearbook, there were severe outbreaks of brown planthopper nation wide in the years 1966, 1969, 1973, 1977, 1983 and 2003 and extremely severe outbreaks in the years 1987, 1991, 2005, 2006 and 2007. The harmed area accounts for more than 50% of the total rice cultivation and it caused a great loss to the rice production of China. Since the harm caused by brown planthopper occurs mainly during rice grain filling and ripening stages, large amounts of pesticide must be applied during this period, which risks contaminating the rice. There remains a need in the industry for a safer way to ensure a high yield from rice cultivation.

Using a brown planthopper resistance gene to breed pest resistance into a rice variety is the most economic and effective method for the integrated control of brown planthopper. The research results of International Rice Research Institute (IRRI) and the practical experience of rice production in Southeast Asia have shown that even rice varieties having medium level resistance are sufficient to control the brown planthopper population so as to have no discernable damage and that no actual harm and loss of yield are caused. Thus, isolating a brown planthopper resistance gene and applying it in the project of rice breeding are the fundamental measures for controlling damage in rice crops caused by brown planthopper.

The study of rice brown planthopper resistance gene began in the 1970s. Up to now, 19 major pest resistance genes have been named (for detailed reviews see Yang H Y et al., 2004 High-resolution genetic mapping at the Bph15 locus for brown planthopper resistance in rice. Theor Appl Genet. 110: 182-191). Among them, the resistance of the three rice varieties (Mudgo, CO22 and MTU15) is controlled by a single dominant gene, this gene is named as Bph1, and another recessive gene bph2, closely linked with Bph1, controls the resistance of rice variety ASD7. In their genetic study of 28 varieties, Lakshminarayana and Khush found that rice variety Rathu Heenati carries a dominant brown planthopper resistance gene Bph3, which is inherited independently from BPh1. In addition, rice variety Babawee contains a recessive gene bph4, which is also inherited independently from bph2. Sidhu and Khush found that Bph3 and bph4 are closely linked, bph4 is also linked with the semidwarf gene sd-1. The genetic analysis of Khush et al about rice variety ARC10550 showed that it contains the recessive gene bph5. In their study of 17 materials resistant to bio-type 4 BPH but sensitive to the other three bio-types, Kabir and Khush found that varieties Swarnalata and T12 contain one pest resistance gene respectively, which are named Bph6 and bph7. The discovery of bph8 and Bph9 is similar to that of the other genes, the recessive gene bph8 is not allelic to bph2 and bph4, the dominant gene Bph9 is not allelic to Bph3 and Bph4. Among the afore-mentioned brown planthopper resistance genes, bph5, Bph6 and bph7 are resistant to brown planthopper bio-type 4, while exhibiting sensitivity to bio-types 1, 2 and 3.

Wild rice is also a source of brown planthopper resistance genes. In 1994, Ishii et al. identified a new dominant brown planthopper resistance gene Bph10 from a transformed line of Australian wild rice (O. australiensis), IR65482-4-136-2-2. This gene is resistant to brown planthopper bio-types 1, 2 and 3. Bph11 is identified from O. eichinger. Rice with a brown planthopper resistance gene can inhibit food fetching, growth and development, and reproduction of brown planthopper, so that the aim of pest resistance is achieved (Hao P Y et al., 2008 Herbivore-induced callose deposition on the sieve plates of rice: an important mechanism for host resistance. Plant Physiology 146: 1810-1820). However, up to now, no rice brown planthopper resistance gene has been cloned.

Map-based cloning is also called positional cloning, which is a gene cloning technology developed along with the development of molecular marker genetic linkage map. The steps of map-based cloning comprise genetic mapping of the target gene, physical mapping, sequence analysis and genetic transformation and test of function. Theoretically, any gene that is able to be positioned can be isolated by map-based cloning. Generally, map-based cloning is suitable for species with relatively small genomes, such as the monocot model plant rice, in which the ratio between genome physical distance and genetic distance is small and has plenty of markers. As a gramineous model plant, rice has a genome that is the center of a concentric circle formed by the genomes of 7 gramineous plants, such as wheat and broomcorn, and it is one of the crops most suitable to use map-based cloning to isolate a target gene. Multiple genes already cloned in rice were cloned by map-based cloning, for example, the Xanthomonas oryzae pv. oryzae resistance gene Xa-21 (Song WY et al. 1995, A Receptor Kinase-Like Protein Encoded by the Rice Disease Resistance Gene, Xa21. Science, 270: 1804-1806), Xa-1 (Yoshimura et al. 1998, Expression of Xa-1, a bacterial blight-resistance gene in rice, is induced by bacterial inoculation. PNAS, 95: 1663-1668) and Xa-26 (Sun et al. 2004, Xa26 a gene conferring resistance to Xanthomonas oryzae pv. oryzae in rice, encodes an leucine-rich repeat LRR receptor kinase-like protein. Plant Journal, 37: 517-527), rice blast resistance gene Pi-b (Wang et al. 1999, The Pi-ta gene for rice blast resistance belongs to the nucleotide binding and leucine-rich repeat class of plant disease resistance genes. Plant Journal, 1999, 19: 55-64) and Pi-ta (Bryan et al. 2000, A single amino acid difference distinguishes resistant and susceptible alleles of the rice blast resistance gene Pi-ta. Plant Cell, 12: 2033-2046), and the tillering gene cloned by Li (Li et al. 2003, Control of tillering in rice. Nature 422: 618-621), salt tolerance gene (Ren et al. 2005, A rice quantitative trait locus for salt tolerance encodes a sodium transporter. Nature Genetics 37(10): 1141-1146) and high yield gene (Weiya Xue et al. 2008. Natural variation in Ghd7 is an important regulator of heading date and yield potential in rice. Nature Genetics 40, 761-767).

### SUMMARY OF THE INVENTION

The aim of the present invention is to provide a rice brown planthopper resistance gene Bph14, which has a nucleotide sequence as shown in SEQ ID NO:1.

Another aim of the present invention is to provide methods of using the brown planthopper resistance gene Bph14 to improve rice breeding.

A further aim of the present invention is to provide methods of using the brown planthopper resistance gene Bph14 to increase the resistance of rice to brown planthopper.

The present invention provides a method of establishing an isolated population of rice resistant to brown planthopper. It uses map-based cloning and isolates rice brown planthopper resistance gene Bph14. Co-segregation marker assay shows that this gene is co-separated with brown planthopper resistance property. By genetic transformation of the Bph14 gene, so that the transformed rice shows the phenotype of brown planthopper resistance, the function of this gene is proved.

The nucleotide sequence of the Bph14 gene of the present invention is as shown in SEQ ID NO:1. The full length of this gene is 9921 bp, containing 1 intron and 2 exons, its CDS are the regions base pairs 3387-7289 and base pairs 7936-8004 respectively. The full length of the cDNA is 3972 bp, encoding for 1323 amino acids, its amino acid sequence is as shown in SEQ ID NO:3. This protein belongs to the family of nucleotide-binding site—leucine-rich repeat NBS-LRR, the active center region of 180-464 is a conservative NB-ARC domain, including conservative P-loop, ATP binding domain and kinase 1a (Van der Biezen E A, Jones J D G. The NB-ARC domain: a novel signalling motif shared by plant resistance gene products and regulators of cell death in animals. 26 Mar. 1998. Current Biology 8(7):R226-R228).

It should be understood, without influencing the activity of the Bph14 protein, the skilled person in the art can substitute, insert and/or delete one or more amino acids of the amino acid sequence as shown in SEQ ID NO:3 to make an amino acid sequence having the same function.

Besides, considering the degeneracy of codons, for example, the gene sequence coding for the above-mentioned protein can be modified in its coding region without changing the amino acid sequence or in the non-coding region without affecting protein expression. Therefore, the present invention also includes a nucleotide sequence with one or more nucleotide substituted, inserted and/or deleted from the gene sequence coding for the above protein and having the same function as the above coding gene. The present invention also comprises sense sequence or antisense sequence derived from the gene, including cloning vector or expression vector containing the nucleotide sequence or its fragment, host cell containing the vector, a transformed plant cell and a transgenic plant containing the nucleotide sequence or a fragment thereof.

The skilled person in the art will understand that molecular markers designed or made according to the published sequence of the present invention can be used for the breeding of brown planthopper resistant rice.

The Advantage and Effect of the Present Invention:

1.The successful cloning of the Bph14 gene further proved the ability of map-based cloning in cloning important rice genes. Genes cloned using this method have clear functions and beneficial effects.

2. Although in rice, multiple genes coding for nucleotide-binding site NBS structure containing proteins have been cloned, most of them are related to disease resistance. The Bph14 gene cloned in the present invention has the evident property of brown planthopper resistance and this is of great importance for fully understanding the biological functions for genes of this type.

3. To date, no rice brown planthopper resistance gene is known to have been cloned, and the molecular mechanism of resisting brown planthopper in rice remains unclear. The Bph14 gene cloned in the present invention can increase the resistance of rice against brown planthopper, which will promote research to understand the molecular mechanisms of brown planthopper resistance in rice.

4. Bph14 dramatically increases the brown planthopper resistance of rice. Using Bph14 for rice breeding via genetic transformation or cross-breeding can improve the resistance of rice against brown planthopper, so that the harm caused by brown planthopper is alleviated and the aim of increasing and stabilizing the yield is achieved.

5. The piercing-sucking insect is a detrimental pest to the agricultural industry. The cloning of the Bph14 gene and the verification of its brown planthopper resistance function serve as an important reference for studying resistance in other plants to other classes of piercing-sucking insects.

By using primers provided (SEQ ID NOs: 14-27), brown planthopper resistance may be determined. The existence of brown planthopper resistance gene Bph14 in rice variety B5 is indicated if 222 base pair (bp) fragments (SG1) can be amplified using SEQ ID NOs: 14 and 15; or 221 bp fragments (SG6) can be amplified using SEQ ID NOs: 16 and 17; or 227 bp fragments (SG9) can be amplified using SEQ ID NOs: 18 and 19; or 158 bp fragments (RM570) can be amplified using SEQ ID NOs: 20 and 21, or 230 bp fragments (SM1) can be amplified using SEQ ID NOs: 22 and 23; or 172 bp fragments (76-2) can be amplified using SEQ ID NOs: 24 and 25; or 218 bp fragments (SM4) can be amplified using SEQ ID NOs: 26 and 27; wherein the Bph14 gene locates between marker SG1 and SM4 in the end of the long arm of the 3^{rd} chromosome in rice genome. We have used SG1 and SM4 to screen 3700 single plants from the F₂ population and 5000 single plants from the F₅ population, and have acquired single plants that have recombinant molecular markers between SG1 and SM4. Based on the genotype of these recombinant single plants and the resistance grade of their corresponding lines, molecular marker 76-2 cosegregates with brown planthopper resistance gene Bph14, and molecular marker SG1, SG9, SG6, RM570, SM1 and SM4 all can be used to screen brown planthopper resistant rice varieties carrying Bph14 gene.

One aspect of the present invention provides an isolated nucleic acid molecule comprising a nucleotide sequence that comprises a brown planthopper resistance gene Bph14 selected from the group consisting of SEQ ID NO: 1 and SEQ ID NO: 2. In another aspect, the nucleotide sequence encodes a polypeptide molecule comprising the amino acid sequence SEQ ID NO: 3. In yet another aspect, the nucleotide sequence is operably linked to a heterologous promoter.

Another aspect of the present invention provides an expression vector comprising the isolated nucleic acid molecule comprising a nucleotide sequence that comprises a brown planthopper resistance gene Bph14 selected from the group consisting of SEQ ID NO: 1 and SEQ ID NO: 2. In yet another aspect, the present invention provides a transgenic plant, plant tissue, or plant cell comprising the expression vector. In still yet another aspect, the transgenic plant, plant tissue, or plant cell is a monocot. In further yet another aspect, the transgenic plant, plant tissue, or plant cell is rice.

Yet another aspect of the present invention provides a method for producing a transgenic plant which expresses a Bph14 gene, comprising the steps of: (a) stably transforming a cell of a plant with a nucleic acid molecule comprising a nucleic acid sequence selected from the group consisting of SEQ ID NO: 1 and SEQ ID NO: 2 to produce a transformed cell; (b) regenerating a transgenic plant from the transformed cell; and (c) growing the transgenic plant wherein the nucleic acid molecule is expressed. In another aspect, the transgenic plant is a monocot. In still yet another aspect, the transgenic plant is rice.

Further yet another aspect of the present invention provides a molecular marker associated with brown planthopper resistance, wherein the molecular marker is selected from the group consisting of: SG1, SG6, SG9, RM570, SM1, 76-2, and SM4. In one aspect, SG1 is amplified by primers SEQ ID NOs: 14 and 15. In another aspect, SG6 is amplified by primers SEQ ID NOs: 16 and 17. In yet another aspect, SG9 is amplified by primers SEQ ID NOs: 18 and 19. In still yet another aspect, RM570 is amplified by primers SEQ ID NOs: 20 and 21. In further yet another aspect, SM1 is amplified by primers SEQ ID NOs: 22 and 23. In another aspect, 76-2 is amplified by primers SEQ ID NOs: 24 and 25. In yet another aspect, SM4 is amplified by primers SEQ ID NOs: 26 and 27.

Still yet another aspect of the present invention is a method for determining the presence or absence of brown planthopper resistance in a plant or seed, comprising analyzing genomic DNA from the plant or seed for the presence of a molecular marker linked to a quantitative trait locus associated brown planthopper resistance, wherein the molecular marker is selected from the group consisting of: SG1, SG6, SG9, RM570, SM1, 76-2, and SM4. In another aspect, the method further comprises analyzing genomic DNA from a plant or seed for the presence of a second molecular marker linked to a quantitative trait locus associated with brown planthopper resistance, wherein the second molecular marker is G1318. In yet another aspect, the plant or seed is a monocot. In still yet another aspect, the plant or seed is rice.

Another aspect of the present invention is a quantitative trait locus associated with brown planthopper resistance, wherein the quantitative trait locus is located in a 34 kb region between a first molecular marker and a second molecular marker on chromosome 3 of rice. In another aspect, the quantitative trait locus comprises Bph14.

### BRIEF DESCRIPTION OF THE DRAWINGS

FIG. 1. The positioning of the brown planthopper resistance major gene Bph14 in the 3r^{d} chromosome of rice variety B5. A, the scanning result of QTL. Horizontal lines represent the 3r^{d} chromosome of rice, and perpendicular short lines represent molecular markers in the chromosome. The values between markers indicate the genetic distance (cM) between markers. Triangles represent the LOD value of each marker. LOD higher than 2.0 represents the existence of one QTL. “n” is the number of single plants in the population; B, the result of F₂ recombinant single plants screening using SG1 and SM4. The results from phenotypes and genotypes are integrated, and Bph14 cosegragates with molecular marker SM1. The values below the markers represent the number of recombinant single plants between the molecular marker and Bph14. n represents the number of total F₂ single plants screened; C, the result of F₅ recombinant single plants screening using RM570 and SM4. Bph14 cosegregates with molecular marker 76-2. The values below the markers represent the number of recombinant single plants between the molecular marker and Bph14. n represents the number of total F₅ single plants screened; D, 76B10 is a BAC clone of B5 genome library. Based on the comparison between its sequence and Nipponbare sequence, 76-2, the primer designed based on the difference between the sequences, cosegregrates with Bph14. Bph14 is positioned in the 34 kb region between SM1 and G1318.

FIG. 2. The electrophoresis graph of single plants examined with the SSR marker 76B10-2. The first two lanes are the pest-resistant parental plant RI35 and the pest-sensitive plant TN1, the rest shows the genotypes of single plants of the F2 population.

FIG. 3. Mapping of Bph14. A: The result of Bph14 preliminary mapping. The name of the markers are above the chromosome, the numbers represent the genetic distance (cM) between the markers, the QTL scanning result shows that there is a biggest LOD value 49.3 between the molecular markers R1925 and G1318.

B: Results of fine mapping. The numbers between the molecular markers represent the number of single plants with the recombination of the marker and Bph14. Bph14 is between the molecular markers SM1 and G1318.

C: The physical map between SM1 and G1318, Bph14 is located in the 34 kb region between SM1-G1318.

### DESCRIPTION OF SEQUENCE LISTING

SEQ ID NO:1 is the nucleotide sequence of Bph14 gene.

SEQ ID NO:2 is the Bph14 cDNA sequence.

SEQ ID NO:3 is the Bph14 protein sequence encoded by SEQ ID NO: 1.

SEQ ID NO:4 and 5 and SEQ ID NO:6 and 7 are pairs of primers used to amplify the Bph14 gene from the genome of B5.

SEQ ID NO:8 and 9 is a pair of primers used to amplify the cDNA of Bph14 gene.

SEQ ID NO:10 and 11 is a pair of primers used to amplify 35S promoter from pCAMBIA1301.

SEQ ID NO:12 and 13 are labeling primers of the Bph14 gene.

SEQ ID NO:14 and 15 are primers for molecular marker SG1.

SEQ ID NO:16 and 17 are primers for molecular marker SG6.

SEQ ID NO:18 and 19 are primers for molecular marker SG9.

SEQ ID NO:20 and 21 are primers for molecular marker RM570.

SEQ ID NO:22 and 23 are primers for molecular marker SM1.

SEQ ID NO:24 and 25 are primers for molecular marker 76-2.

SEQ ID NO:26 and 27 are primers for molecular marker SM4.

### DETAILED DESCRIPTION OF THE INVENTION

The following embodiments further illustrate the contents of the present invention, but they should not be understood to limit the present invention. Modifications or substitutions made to the method, process or condition of the present invention, when not deviating from the spirit and essence of the present invention, all are within the scope of the present invention.

If not specifically indicated, the technical means used in the embodiments are routine means well known to the skilled person in the art.

### EXAMPLES

### Example 1: Positional Cloning of the Bph14 Gene

1.1.Preliminary Mapping Result of Bph14

The brown planthopper resistant rice material RI35 (Hao P Y, Liu C X, Wang Y Y, Chen R Z, Tang M, Du B, Zhu L L, He G C (2008) Herbivore-induced callose deposition in the sieve plates of rice: an important mechanism for host resistance. Plant Physiology 146: 1810-1820)was crossed with a rice variety sensitive to brown planthopper (Taichung native 1, TN1, bought from national rice seed resource library) to establish the F2 population containing Bph14. In order to evaluate the brown planthopper resistance phenotype of each single plant in the F2 mapping population, the seedling bulk screening test was used to examine the resistance of each single plant in the population. The pest resistance level of single F2 plant is calculated according to the pest resistance level of all single plants of the corresponding F₂₋₃ family. Using the methods of PCR (polymerase chain reaction), polyacrylamide gel electrophoresis, RFLP probe and Southern blotting (Sambrook, et al.) the separation state of SSR and RFLP molecular probes was detected of each single F2 plant. Based on the subtype of F2 molecular marker, JoinMap3.0 software (Kyazma B. V., PO Box 182, 6700 AD Wageningen, Netherlands) was used to establish the molecular marker genetic linkage map of the rice chromosome. With the assistance of the quantitative character analyzing software MapQTL5.0 (Kyazma B. V., PO Box 182, 6700 AD Wageningen, Netherlands), interval mapping analysis was combined with the quantitative data of brown planthopper resistance phenotype collected in the seedling bulk screening test. The results indicate: a QTL peak value exists between the 3^{rd} chromosome molecular markers R1925 and G1318, the LOD value reaches 49.3 and the contribution rate to the phenotypic variance is 90.6%.

1.2 Fine Mapping of Bph14

Based on earlier results, the methods of PCR (polymerase chain reaction) and polyacrylamide gel electrophoresis are used to screen the F2 population with two SSR markers RM514,located outside of R1925 and G1318, and SM1, located within R1925 and G1318, to get 54 recombined single plants. Integrating the molecular markers of recombinant single plants, the single plants having identical molecular markers and the same level of pest resistance were pooled (Table 1). Except from the single plant SA69, the 12 single plants RT25-RT15 have identical phenotype with the molecular marker SM1, but in SA69, the phenotype is identical with G1318. Thus, Bph14 is located between SM1 and G1318.

TABLE 1
The pest resistance performance of recombined F2 single plants

| [symbol] | RM514 | SG1 | R1925 | SG9 | SG6 | RM570 | SM1 | G1318 | [symbol] | [symbol] |
|---|---|---|---|---|---|---|---|---|---|---|
| RT1 | R | R | R | R | R | R | H | H | H | 5.6 |
| RT5 | R | R | R | R | R | H | H | H | H | 4.74 |
| RT16 | R | R | H | H | H | H | H | H | H | 5.83 |
| RT2 | R | H | H | H | H | H | H | H | H | 5.49 |
| SA50 | R | H | H | H | H | H | R | R | R | 3.93 |
| SA74 | H | H | H | H | H | H | R | R | R | 3.96 |
| SA55 | S | H | H | H | H | R | R | R | R | 3.86 |
| RT18 | H | H | H | H | H | R | R | R | R | 4.04 |
| RT83 | H | H | H | H | R | R | R | R | R | 4.56 |
| RT82 | H | H | H | R | R | R | R | R | R | 4.1 |
| RT10 | H | R | R | R | R | R | R | R | R | 4.43 |
| RT25 | H | H | H | S | S | S | H | H | H | 4.88 |
| SA51 | H | H | S | S | S | H | H | S | H | 4.48 |
| SA66 | H | H | S | S | S | H | H | S | H | 4.78 |
| SA69 | S | S | S | S | S | H | H | S | S | 7.38 |
| RT84 | H | H | H | H | H | H | S | S | S | 7.23 |
| RT24 | H | H | H | H | H | S | S | S | S | 8.55 |
| SA60 | H | H | H | S | S | S | S | S | S | 7.55 |
| RT3 | H | S | S | S | S | S | S | S | S | 8.25 |
| SA102 | S | S | S | S | S | S | S | H | S | 8.32 |
| RT8 | S | S | S | S | S | S | H | H | H | 5.58 |
| RT7 | S | S | S | S | S | H | H | H | H | 5.39 |
| RT17 | S | S | S | S | H | H | H | H | H | 4.35 |
| RT15 | S | H | H | H | H | H | H | H | H | 4.96 |

R = resistant,
H = heterozygous,
S = susceptable.
The top axis of Table 1 indicates the molecular marker screened. [symbol]
indicates data missing or illegible when filed

1.3. Construct the Brown Planthopper Resistance Genomic Library

For the preparation of plant high molecular weight genomic DNA, see the methods of Zhang Hongbin et al.(Zhang et al., Preparation of megabase DNA from plant nuclei Plant J 1995, 7, 175-184). Nuclei from the young leaves of brown planthopper resistance rice B5 (Wang B N, Huang Z, Shui L H, Ren X, Li X H, He G C (2001) Mapping of two new brown planthopper resistance genes from wild rice. Chinese Science Bulletin 46: 1092-1095) was extracted and embedded in low-melting point agarose. An appropriate amount of restriction enzyme BamHI was added to the embedded nuclei for partial digestion. Pulsed field gel electrophoresis was performed with the CHEF Mapper pulsed field electrophoresis system to isolate the needed fragment. The strongest gel band from the region containing the 50-250 kb fragment was cut out and placed into the dialysis bag. The DNA fragment was recovered using electroelution (Strong et al., Marked improvement of PAC and BAC cloning is achieved using electroelution of pulsed-field gel-separated partial digests of genomic DNA. Nucleic Acids Res. 1997, 25, 3969-3961). The large fragment DNA isolated with electroelution was collected and put into a 1.5 ml centrifuge tube, 600 ng recovered DNA fragment (50-250 Kb) was mixed with 200 ng dephosphorylated vector BIBAC2, incubated at 60° C. for 10 min and cooled to room temperature. T4 DNA ligase was added and the mixture was incubated at 16° C. for 16 h. Taking 2 μl ligation product and 40 μl DH10B competent cells, the Gene Pulser system was used to perform electrotransformation. The transformed cells were plated onto agarose containing 50 mg/l Kanamycin and incubated at 37° C. overnight. Positive clones were picked from the plate and inoculated into a 384 well cell culture plate containing 70 μl medium, and incubated at 37° C. for 30 h. After the construction of the library, two copies were made with Genetix Q-PIX and one perserved at −80° C. In order to estimate the distribution of the length of the inserted fragment and the volume of the clones, 30 BIBAC clones were randomly picked from the library, and their plasmids extracted by alkaline lysis. After digesting with appropriate amount of NotI, the length of the inserted fragment was confirmed with pulsed field gel electrophoresis (Shi Z Y, Ren X, Weng Q M, Li X H, He G C (2003) Construction of genomic library of a BPH-resistant rice line with binary vector and physical map of Qbp1 locus. Plant Science 1165:879-885).

1.4. Construction of the Physical Map of the SM1-G1318 Region.

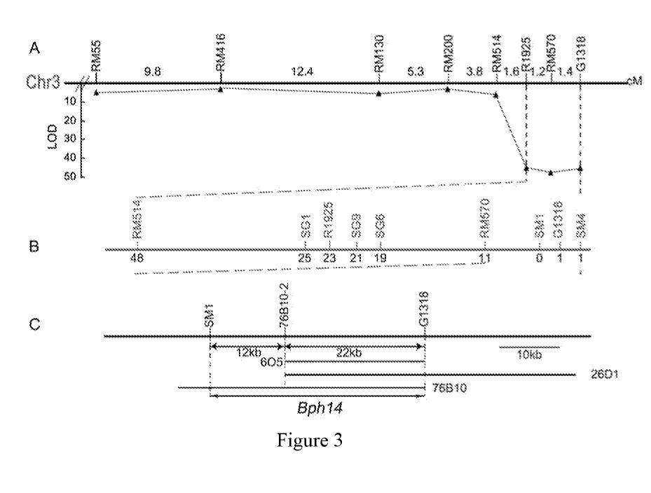

All the BAC clones were screened for the R1925-G1318 region. After double digestion with BamHI and EcoRI, electrophoresis was performed and the nucleic acid fragments were transferred to a membrane. Then, the end of the digested clone was labeled with radioactive α-³²P-dCTP. Southern blotting with the BAC clones was performed as before, and the BAC clones which have overlaps and the length of the overlapped fragment based on the hybridization signal were identified. Based on the results, the physical map was constructed (FIG. 3C). For the terminal isolation of BAC positive clones, see the TAIL-PCR method invented by Liu Yaoguang et al. (Liu and Whittier, Thermal asymmetric interlaced PCR: automatable amplification and sequencing of insert end fragments from P1 and YAC clones for chromosomewalking. Genomics 25: 674-681). The results of the screen and TAIL-PCR show that the BAC clone 76B10 contains the complete Bph14 gene (FIG. 3C).

1.5. Analysis of Candidate Genes in the SM1-G1318 Region

Sequence analysis for the entire sequence of the Bph14 gene containing clone was performed; the NCBI database was searched using this sequence as the target sequence to identify the homologous sequence of the Nipponbare genome in this region. RiceGAAS online software (Sakata,K., Nagamura,Y., Numa,H., Antonio,B. A., Nagasaki,H., Idonuma,A., Watanabe,W., Shimizu,Y., Horiuchi,I., Matsumoto,T., Sasaki,T. & Higo,K.: “RiceGAAS: an automated annotation system and database for rice genome sequence” , 2002. Nucleic Acids Res., 30: 98-102) was used to perform gene prediction and annotation, also ClustalW was used for comparative analysis (table 2).

TABLE 2
Comparison of the predicted pest-resistant rice genes in the region of the Bph14
gene with the predicted genes of Nipponbare

| the predicted genes of Nipponbare | | | | the predicted pest-resistant rice genes | | | | |
|---|---|---|---|---|---|---|---|---|
| | | Number | | | | Number | | |
| | | of | Number | | | of | Number | |
| | | amino | of | | | amino | of | Similarity |
| NO: | Predicted function | acid | exon | NO: | Predicted function | acid | exon | (%) |
| g1 | Putative ARPC | 75 | 2 | g1 | Putative ARPC | 75 | 3 | 100 |
| | protein p20 | | | | protein p20 | | | |
| g2 | Putative B cell | 217 | 3 | g2 | Putative B cell | 189 | 2 | 94.7 |
| | receptor related | | | | receptor related | | | |
| | protein 31 | | | | protein 31 | | | |
| g3 | Putative reverse | 1997 | 4 | g3 | Putative NO | 246 | 4 | 96.6 |
| | transcription | | | | inducing protein | | | |
| | transposon | | | | NOI | | | |
| g4 | Putative NO | 238 | 4 | g4 | putative disease- | 1333 | 2 | 83.4 |
| | inducing protein | | | | resistant protein | | | |
| | NOI | | | g5 | Putative RPM1 | 148 | 4 | 33.8 |
| g5 | Putative disease- | 1315 | 1 | | interacting protein | | | |
| | resistant protein | | | | RIN4 | | | |
| g6 | Unknown protein | 15 | 2 | g6 | Putative disease- | 1121 | 3 | 99.6 |
| g7 | Unknown protein | 132 | 2 | | resistant protein | | | |
| g8 | Putative RPM1 | 168 | 3 | g7 | Putative protein | 148 | 3 | |
| | interacting protein | | | g8 | Putative protein | 115 | 3 | |
| | RIN4 | | | g9 | Unknown protein | 223 | 3 | |
| g9 | Unknown protein | 49 | 2 | g10 | putative disease- | 680 | 2 | 99.7 |
| g10 | Putative protein | 87 | 2 | | resistant protein | | | |
| g11 | Putative disease- | 680 | 2 | | | | | |
| | resistant protein | | | | | | | |

By comparing the predicted genes of the two, it was found that the disease resistant protein encoded by the 4^{th} gene of the pest resistance rice is quite different from that of Nipponbare. Now, it is commonly considered that the sucking and eating of rice by piercing-sucking insects is similar to the process of rice infection by pathogenic bacteria, therefore, the mechanism of rice to resist piercing-sucking insects might be the same as that of resisting pathogenic bacteria. Thus, this gene can be determined to be Bph14.

1.6. Screening the cDNA Library

Using the predicted gene corresponding to the EST as a probe, phage in situ hybridization with the cDNA library of brown planthopper induced pest-resistant rice B5 was performed (Wang X L, Weng Q M, You A Q, Zhu L L, He G C (2003) Cloning and characterization of rice RH3 gene induced by brown planthopper. Chinese Science Bulletin 48: 1976-1981). After three rounds of in situ hybridization, two chosen phage clones with PCR were examined, and afterwards the length of the inserted fragment was determined with enzyme digestion. Full length cDNA was sequenced. Its nucleotide sequence is as shown in sequence listing, SEQ ID NO:2. However, the skilled person in the art will understand that according to the nucleotide sequence disclosed in the present invention, by designing appropriate primers, the Bph14 gene can be amplified and obtained from the genome of brown planthopper resistance rice. For example, primers: 5′ctccctgactgaagaagagaagag3′ (SEQ ID NO: 4) and 5′tgctagctgtgattctcttatgatg3′ (SEQ ID NO: 5), the sequence can be obtained by using long fragment PCR amplification kit and amplifying the genome of brown planthopper resistance rice or wild rice (94° C. for 2 minutes; 30 cycles of 94° C. for 15 seconds, 58° C. for 30 seconds, 72° C. for 7 minutes; 72° C. for 2 minutes).

### Example 2: Functional Verification of Bph14 and Its Application

2.1. Construction of Genetic Transformation Vector

The vector used is pCAMBIA1301 (bought from Australia Center for the Application of Molecular Biology to International Agriculture). Based on the result of genome sequencing, primers were designed (5′cggaattcctccctgactgaagaagagaagag3′ (SEQ ID NO: 6), 5′cggaattctgctagctgtgattctcttatgatg3′ (SEQ ID NO: 7) that contain an EcoRI linker. Using these primers, the genome of pest-resistant rice B5 was amplified as described below(Z. Huang et al.,

Identification and mapping of two brown planthopper resistance genes in rice. Theor Appl Genet, 2001, 102: 929-934). The total volume of PCR reaction is 50 μl: 1 μl DNA, 10× buffer 5 μl, 10 mM dNTP 1 μl, 10 mM primers each 3 μl, high-fidelity Taq enzyme 1U; reaction program: 94° C. 2 min, 94° C. 15 s, 58° C. 30 s, 72° C. 7 min30 s, totaling 30 cycles. The product was purified by adding 110 volume 3 mM NaAC and 2× volume absolute alcohol. The obtained sequence contains a 1960 bp promoter and 4997 bp genomic sequence upstream of Bph14, and downstream 436 bp 3′ non-translational region, which was digested with EcoRI, where the total volume the digestion system was 20 μl: about 5 μl (1 μg) PCR product, 1× reaction buffer, EcoRI 1U, mixed well and incubated at 37° C. overnight. The product was precipitated with 110 volume 3 mM NaAC and 2× volume absolute alcohol, recovering the needed fragment. The digestion system of pCAMBIA1301 vector is as stated before, purified with the purification kit. The ligation reaction used is as follows: genomic fragment 1 μl vector 0.5 μl , 2U T4 ligase, 5× buffer 2 μl, total volume 10 μl, ligate at 4° C. overnight. The ligation product was transformed into E.coli DH10B by heat shocking at 42° C. for 90 s, adding in 400 μl LB, recovering for 45 minutes, transfering 200 μl, of the culture onto LA plate containing kanamycin, and incubating at 37° C. overnight. Single clones were picked, amplified, and plasmid extracted and tested by enzyme digestion. A positive clone was picked and electro-porated into Argobacterium EHA105. Cloning was confirmed by extracting the plasmid and verified with PCR. The Argobacterium culture containing the constructed vector was preserved by taking 750 μl, and adding 50% glycerol of the same volume, mixing well. The culture was stored at −70° C.

Primers were designed based on full length cDNA sequence, containing XmaI and XbaI linker (5′tccccccgggatggcggagctaatggccac3′(SEQ ID NO: 8), 5′gctctagactacttcaagcacatcagccta3′ (SEQ ID NO: 9)). Total RNA was extracted from B5 leaf sheath using TRIzol of Invitrogen (Invitrogen Corporation, 5791 Van Allen Way, PO Box 6482, Carlsbad, Calif. 92008), then, the cDNA of B5 was obtained by using the reverse transcription kit of Fermentas (Fermentas International Inc, 830 Harrington Court, Burlington Ontario L7N 3N4 Canada); reaction system: total RNA 1 μg, oligo(dT) 1 μl, 5×buffer 4 μl, inhibitor 1 μl, 10 mMdNTP 2 μl, reverse transcriptase 1 μl, incubate at 42° C. for 1 hour. B5 cDNA was amplified using the designed primers. The PCR reaction system is as described above, however, in the program, elongate at 72° C. for 4 min to get the cDNA sequence of Bph14 . Meanwhile, the promoter required for cDNA transcription can be obtained from PCR amplification of the 35S promoter present in pCAMBIA1301. Using the designed primers containing EcoRI and XmaI linker (5′cggaattcatggtggagcacgacactct3′ (SEQ ID NO: 10), 5′tccccccgggatctcattgccccccgggat3′ (SEQ ID NO: 11)), the 35S promoter sequence was amplified from pCAMBIA1301. The PCR reaction system used is as described above, however the elongation time is 1 min. The 35S promoter and the pCAMBIA1301 vector were digested with EcoRI and XmaI each. The 35S fragment and the linearized vector were ligated and transformed into E. coli after recovery. The obtained positive clone and the Bph14 cDNA sequence was digested with XmaI and XbaI each, the products were recovered, ligated and transformed. A 35S:Bph14 vector was constructed and electro-porated into Argobacterium EHA105, the detailed process is described above.

2.2 Genetic Transformation

The above mentioned Bph14 genomic transformation vector and cDNA transformation vector were separately introduced into the ordinary rice variety Kasalath (bought from national rice seed resource library or national rice research institute) sensitive to brown planthopper using the genetic transformation method mediated by Argobacterium EHA 105(Hiei et al., 1994,

Efficient transformation of rice (Oryza sativa L.) mediated by Argobacterium and sequence analysis of the boundaries of the T-DNA. Plant Journal 6:271-282). At the same time, a blank vector (pCAMBIA1301) was used as a negative control.

2.3 The Expression Result of Bph14 Gene and its Application

14 cultured seedlings, obtained from each of the two transformed lines above, and 4 control seedlings, were planted in the field. After harvesting the T1 generation separately, homozygous plants (14 plants each) were selected for the pest resistance test. After the pest resistance test at the seedling stage and at mature stage, in both cases, the brown planthopper resistance of transgenic plants is evidently increased, while the control plants have no resistance against brown planthopper. All the pest resistance level of transgenic rice at seedling stage is between Grade 3-5, as determined by the process set forth in Huang, et al. (Huang Z et al, 2001 Identification and mapping of two brown planthopper resistance genes in rice. Theor. Appl. Genet. 102, 929-934). The transgenic plants at mature stage are in good condition after the addition of pests and they can set seeds normally. At the same time, EPG (Peiying Hao et al, Herbivore-induced callose deposition on the sieve plates of rice: an important mechanism for host resistance. PlantPhysiol, 2008, 146: 1810-1820) showed that when brown planthoppers feed on transgenic plants, evidently less time is spent on phloem. The test of honeydew method (P. Paguia, Honeydew excretion measurement techniques for determining differential feeding activity of biotype of Nilaparvata lugens on rice varieties. J. Econ. Entomol, 1980, 73: 35-40) proved that the amount of excretion egested by brown planthopper fed on transgenic plants decreased. Thus, the cloned Bph14 can cause resistance of the rice against the feeding of brown planthoppper on rice.

### Example 3: Molecular Marker Assists the Selection of Bph14 Carrying Brown Planthopper Resistance Rice

3.1 Based on the genomic sequence and cDNA sequence of Bph14 gene, multiple pairs of primers of SSR marker or STS marker can be designed. In the present embodiment, the pair of primers 5′ctgctgctgctctcgtattg3′ (SEQ ID NO: 12), 5′ cagggaagctccaagaacag3′ (SEQ ID NO: 13) is used as labeling primers for the selection of rice with pest resistance. The length of the amplified fragment is 172 bp. By performing PCR amplification, using primers designed on the Bph14 gene sequence, one can test for the presence of the molecular marker by polyacrylamide gel electrophoresis. The cross-breeding offspring plants showing the same PCR bands as pest-resistant rice (amplification product contains a 172 bp fragment) are the selected plants containing Bph14 gene (FIG. 2). The pest resistance of these plants is confirmed with seedling bulk screening test and test at mature stage. Brown planthopper resistance rice is bred through self cross and economical character selection of these plants.

3.2 One can evaluate brown planthopper resistance of the mapping population by using the seedling bulk screening test: F3 seeds were harvested from the F2 plants, and approximately 20 seedlings (called one family) were grown in a tray. Resistant control variety RI35 and sensitive control variety TN1 were grown together. Once the plants developed approximately 2-3 leaves, the plants were inoculated with 2nd-4th instar brown planthopper nympha (10 nymphaplant)and the state of damage was recorded in each of the families when all sensitive control TN1 plants were dead. The experiment was repeated for 3 times with each material. According to the results of pest resistance evaluation, the families of the mapping population were classified as to their pest resistance level.

### Example 4: More Molecular Markers for Determining Brown Planthopper Resistance Genotype

4.1. The Construction of RI35TN1 F₂ Population and Phenotype Evaluation

Using art recognized methods (Wang BN et al , 2001 Mapping of two new brown planthopper resistance genes from wild rice. Chinese. Sci. Bull. 46, 1092-1095, Huang Z et al, 2001 Identification and mapping of two brown planthopper resistance genes in rice. Theor. Appl. Genet. 102, 929-934), the dominant brown planthopper resistance gene Bph14 was found to be located at the end of the long arm of the rice 3^{rd} chromosome, and its RFLP marker is between R1925 and G1318. Due to the high difficulty of the RFLP technique, a huge amount of work is required in large-scale breeding and screening.

In order to search for simple and efficient molecular markers that had tighter link with Bph14, we chose the brown planthopper resistant variety RI35 which originated from the 7^{th} generation of recombinant inbred line between B5 and Minghui 63, only carrying brown planthopper resistance major gene Bph14 (Ren X et al, 2004 Dynamic mapping of quantitative trait loci for brown planthopper resistance in rice. Cereal. Res. Commun. 32, 31-38). Hybrids were produced using RI35 as the female parent and brown planthopper susceptible rice variety. TN1 as the male parent. RI35TN1 F₂ segregation population was constructed. RI35TN1 F_{2:3} lines were respectively obtained from each F₂ single strain by inbreeding.

Resistance evaluation of parent plants and F_{2:3} lines was conducted with introduction during seedling stage. To ensure that the parent plants and each line from the F_{2:3} population grow at the same rate, all experimental materials were respectively soaked and hastened to germinate before the seeding. 20 seeds from each line (variety) were seeded in a 54 cm long, 35 cm wide and 8 cm high bread box filled with nutrient soil. 40 materials were seeded in each box, including 2 resistant parent plants and 4 susceptible parent plants. Thinning was conducted seven days after seeding. Sick and weak seedlings were discarded, and at least 15 plants were kept in each cup. When the seedlings reached three-leaf stage, they were inoculated with 2-3 instar brown planthopper larvae at the ratio of 8 per seedling, and were covered with nylon mesh. When the susceptible variant TN1 died out, each single strain was evaluated for resistance at grade 0, 1, 3, 5, 7 and 9 (Table 3) according to the method described by Huang et al (Huang Z et al, 2001 Identification and mapping of two brown planthopper resistance genes in rice. Theor. Appl. Genet. 102, 929-934), and the resistance grade of each line from the parent plants and the population was calculated by weighted mean, and the single strain genotype was estimated from the resistance grade.

TABLE 3
The classing criteria of brown planthopper resistance
and susceptibility used in the present study

| | Severity of Injury (Evaluated when | Resistance |
|---|---|---|
| Grade | more than 90% Taichung native 1 died) | Level |
| 0 | Healthy plant, no injured leaf | Resistant (R) |
| 1 | One yellow leaf | Resistant (R) |
| 3 | One or two yellow leaves, or one | Medium |
| | withered leaf | Resistant (MR) |
| 5 | Two or three yellow leaves, or two | Medium |
| | withered leaves | Resistant (MR) |
| 7 | Three or four withered leaves, but | Susceptible (S) |
| | plant still alive | |
| 9 | Plant dead | Susceptible (S) |

4.2. Molecular Marker Analysis of RI35TN1 F₂ Population

DNA of the parent plants and each line of F₂ population was extracted using CTAB technique (Murray M G & Thompson, 1980 Rapid isolation of high-molecular-weight plant DNA. Nucleic Acids Res 8: 4321-4325).

Since R1925 and G1318 locate respectively in 32G11 and 96M04, BAC clones of Nipponbare rice genome, we conducted a search for SSR motifs in the sequences of these two BAC clone using the search tool SSRIT described by Temnykh, et al. (Temnykh S, DeClerck G, Lukashova A, Lipovich, Cartinhour S, McCouch S. Computational and experimental analysis of microsatellites in rice (Oryza sativa L.): frequency, length variation, transposon associations, and genetic marker potential. Genome Research. 2001. 11(8):1441-1452) with the following parameters: maximum motif length was tetramer, the minimum repeat was 5. All SSR motifs longer than 15 bases (motif length×repeat times) were selected and primers were designed based on their flanking sequences as candidate SSR markers.

SSR markers were analyzed in accordance with Temnykh's method (Temnykh S et al, 2000 Mapping and genome organization of microsatellite sequences in rice. Theor Appl Genet 100: 697-712). The 10 μL reaction system included: 10 mM Tris-HCl ph8.3, 50 mM KCl, 1.5 mM MgCl₂, 50 μM dNTPs, 0.2 μM primer, 0.5U Taq polymerase and 20 ng DNA template. Amplification is conducted using PTC-100 PCR amplifier: 94° C. 2 min; 94° C. 15 sec, 55° C. 30 sec, 72° C. 1.5 min, 35 cycles; 72° C. 5 min. Amplified products were separated using 6% undenatured PAGE gel, and visualized by silver staining (Zhu et al, 2004 Identification and characterization of a new blast resistance gene located on rice chromosome 1 through linkage and differential analyses. Phytipathology 94:515-519). Amplified DNA bands were observed using a transilluminator with a fluorescent lamp. The results were recorded. Primers that had polymorphism between parent plants were analyzed in F₂ population and population genotype data were obtained.

The genetic map of rice SSR markers was constructed with population genotype data based on the law of linkage and crossover. The software used was MAPMAKER/EXP3.0.

A whole genome scan was conducted using composite interval mapping (CIM) from Windows QTL Cartographer V2.0 software. A segregation analysis between the brown planthopper resistance and SSR markers was conducted using the analytical software MAPMAKER/EXP3.0, and Kosambi functions were converted into genetic distances (cM).

4.3 Screening of RI35TN1 F₂ and F₅ Population Using Molecular Markers and Positioning of Bph14 Gene

Based on the positioning results of QTL, F₂ single plants were screened using the flanking SSR markers SG1 and SM4 to obtain the single plants which had recombination between the two markers. The genotype and phenotype of each single strain were checked as described above to explore which markers cosegregated with the resistance phenotype.

Using molecular marker-assisted selection, we selected F₂ single plants which were heterozygous in Bph14 site and preferably derived from TN1 or heterozygous in other sites; After inbreeding, single plants that were heterozygous in Bph14 site and preferably derived from TN1 in other sites were obtained using molecular marker-assisted selection. Eventually, F₅ inbred population was constructed, in which except for the Bph14 site, all other regions were from the genome of TN1. Based on the results of (1), F₅ single plants were screened using the flanking SSR markers RM570 and SM4 to obtain the single plants which had recombination between the two markers. The genotype and phenotype of each single strain were checked as described above to explore which markers cosegregated with the resistance phenotype.

Based on the results of (2), gene library of B5 was screened, and BAC clones of B5 gene library covering the two markers were obtained. After sequencing, the said sequence was compared for DNA difference with the corresponding sequence of Nipponbare. Primers were designed based on the difference of sequences to amplify the DNA sequence of RI35 and TN1. Primers that have polymorphism were used in the analysis of F₂ and F₅ recombinant single plants to explore whether they cosegregated with resistance phenotype.

4.4 Results and Analysis

Group introduction test in seedling stage showed that the resistance grade of RI35 and TN1 were 2.7 and 9 respectively, which indicated that RI3 5 was brown planthopper resistant while TN1 was susceptible. The resistance grade of F₁ plants was 3.4, showing resistance against brown planthopper, indicating that the resistance of RI35 was controlled by dominant gene. The frequency distribution of the resistance grade of 100 F_{2:3} lines against brown planthopper showed continuous distribution. The minimum value was 3.0 while the maximum value was 9.0, and three obvious peaks were found at the three locations of 3.5, 5.5 and 8.5. Based on the resistance grade F_{2:3} lines were divided into three phenotypes: resistance, segregation of resistance and susceptibility, and susceptibility. The corresponding genotypes of the F₂ single plants were recorded as three types: RR (homozygous resistance), Rr (heterozygous resistance) and rr (homozygous susceptibility). The segregation of resistance and susceptibility of F₂ population was in accordance with a 1:2:1 ratio (χ²=0.54, χ²_{0.05}=5.99) (Table 2).

Huang Zhen and Wang Buna have identified two dominant brown planthopper resistance genes, Bph1 and Bph15, from B5, a fertility line of O. officinalis. RI35 comprises one brown planthopper resistance major gene Bph14. Therefore, in this study, QTL of the F₂ population was positioned using the SSR markers from the 3^{rd} chromosome to determine whether it was in accordance with previous studies.

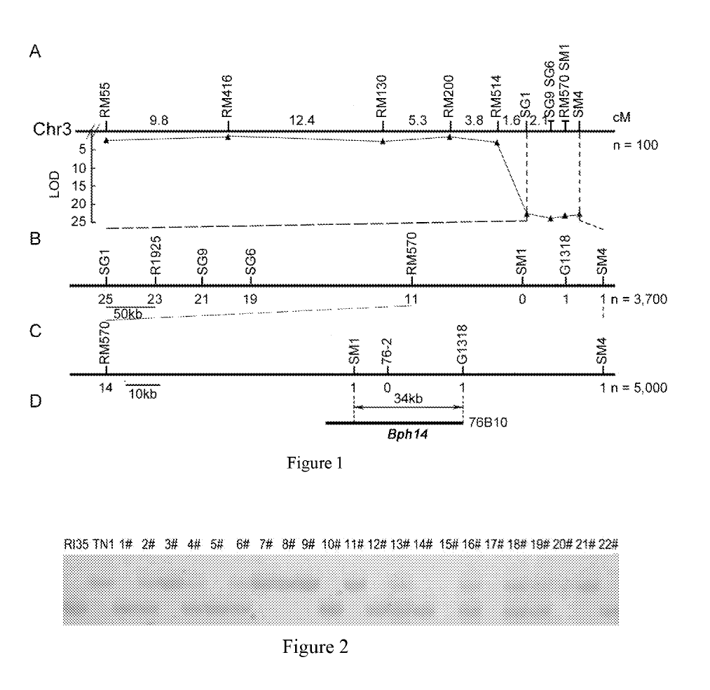

Based on the search results of SSRIT, we selected all the SSR motifs longer than 15 bases (motif length times×repeat times), and designed primers based on their flanking sequences. Depending on the different BAC clones these motifs were situated, these SSR markers were named as SG1, SG2, etc. and SM1, SM2, etc. consecutively. We used these SSR markers to amplify the DNA of the parent plants RI35 and TN1. Only SG1, SG6, SG9 and SM1, SM4 showed polymorphism between parent plants in electrophoresis.

Whereafter we used SSR markers that had polymorphism between parent plants to locate the QTL of the F₂ population. The results showed that there was one QTL site between SG1 and SM4 at the end of the long arm of the 3^{rd} chromosome, whose LOD value was 25.3 and the contribution rate was 67.5%. Molecular marker SG6 and SG9 cosegregated with Bph14. SG1 was 2.1 cM from Bph14; RM570 and SM1 were 0.8 cM from Bph14; SM4 was 1.5 cM from Bph14 (FIG. 1). The accurate rate of SG1, SG6, SG9, RM570, SM1 and SM4 were 98%, 100%, 100%, 99%, 99% and 98%.

The distance between SG1 and SM4 was large. In sequenced indica rice variety Nipponbare, the distance was 270 kb. Therefore, to search for markers more tightly linked to Bph14, we screened 3700 F₂ single plants using SG1 and SM4. The results showed that, only 26 single plants had recombination between marker SG1 and SM4. We used other SSR markers, as well as R1925 and G1318 to check the genotype of the recombinant single plants, and combined with the resistance evaluation results, we found that Bph14 cosegregated with SM1 (Table 5, FIG. 1)

We constructed the inbred F₅ population using the method of molecular marker-assisted selection in which other than Bph14 site, all other regions were from the genome of TN1. 5000 F₅ single plants were screened using the flanking SSR marker RM570 and SM4, and 15 single plants that had recombination between the two markers were obtained. We checked the genotype of the recombinant single plants, and combined with the resistance evaluation results of recombinant single plants, we found Bph14 located between SM1 and SM4. G1318 was used to check the genotype of these recombinant single plants, and eventually Bph14 was positioned between SM1 and G1318 (Table 6, FIG. 1). Through screening the gene library of B5, 76B10, a BAC clone covering both markers was obtained. After sequencing, the sequence was compared for DNA difference with the corresponding Nipponbare sequence, and primers named 76-1, 76-2 etc. were designed based on the difference of sequence to amplify the DNA sequence of RI35 and TN1. Eventually only 76-2 had polymorphism between RI35 and TN1. The obtained single plants were analyzed by 76-2, and it was found that 76-2 cosegregated with Bph14.

The results showed that, the molecular markers described above have few recombinant single plants with Bph14, therefore they are useful to detect the existence of Bph14 resistance major gene, and brown planthopper resistant rice varieties can be obtained using the method of molecular marker-assisted breeding so that the progression of breeding brown planthopper resistant rice varieties in China can be expedited.

TABLE 4
The resistance-susceptibility segregation ratio
against brown plant hoppers in 100 single plants from
RI35/TN1 F₂ segregation population

| | | Corresponding phenotype |
|---|---|---|
| F₂ genotype^{a} | F₂ number of individuals^{b} | of F_{2:3} lines^{c} |
| RR | 23 | RS ≦ 4 |
| Rr | 49 | 4 < RS < 7 |
| Rr | 28 | 7 ≦ RS |

^{a}RR homozygous resistance; Rr heterozygous resistance; rr homozygous susceptible;
^{b}1RR: 2Rr: 1rr Suitability value χ² = 0.54, χ²_{0.05} = 5.99;
^{c}Resistance grade: RS, Resistance Score

TABLE 5
The genotype and phenotype of the F₂ recombinant single plants
screened by molecular markers

| NO: of | | | | | | | | | | |
|---|---|---|---|---|---|---|---|---|---|---|
| Single | | | | | | | | | Pheno- | Resistance |
| Plants | SG1 | R1925 | SG9 | SG6 | RM570 | SM1^{a} | G1318 | SM4 | type | Grade |
| RT1 | R | R | R | R | R | H | H | H | H | 5.6 |
| RT5 | R | R | R | R | H | H | H | H | H | 4.74 |
| RT16 | R | H | H | H | H | H | H | H | H | 5.83 |
| SA50 | H | H | H | H | H | R | R | R | R | 3.93 |
| SA74 | H | H | H | H | H | R | R | R | R | 3.96 |
| RT18 | H | H | H | H | R | R | R | R | R | 4.04 |
| RT83 | H | H | H | R | R | R | R | R | R | 4.56 |
| RT82 | H | H | R | R | R | R | R | R | R | 4.1 |
| SA51 | H | S | S | S | H | H | S | S | H | 4.48 |
| RT84 | H | H | H | H | H | S | S | S | S | 7.23 |
| RT24 | H | H | H | H | S | S | S | S | S | 8.55 |
| SA60 | H | H | S | S | S | S | S | S | S | 7.55 |
| SA102 | S | S | S | S | S | S | H | H | S | 8.32 |
| RT8 | S | S | S | S | S | H | H | H | H | 5.58 |
| RT7 | S | S | S | S | H | H | H | H | H | 5.39 |
| RT17 | S | S | S | H | H | H | H | H | H | 4.35 |

^{a}From this table we can find that the molecular marker SM1 cosegregates with the resistance phenotype. This result shows that Bph14 locates between molecular marker RM570 and G1318 and cosegregates with SM1

TABLE 6
The genotype and phenotype of the F₅ recombinant single plants
screened by molecular markers

| NO: of | | | | | | | |
|---|---|---|---|---|---|---|---|
| Single | | | | | | | Resistance |
| Plants^{a} | RM570 | SM1 | 76-2^{b} | G1318 | SM4 | Phenotype | Grade |
| RT40-9 | S | R | R | R | R | R | 3.63 |
| RT85-1 | S | R | R | R | R | R | 4.56 |
| RT87-4 | R | S | S | S | S | S | 8.66 |
| RT84-5 | R | S | S | S | S | S | 7.75 |
| RT12-5 | R | H | H | H | H | H | 5.65 |
| RT7-8 | S | S | H | H | H | H | 6.07 |
| SA102 | S | S | S | H | H | S | 8.32 |

^{a}Numbers of the single plants indicate that F₅ populations eventually obtained from these F₂ single plants using molecular marker-assisted selection were used to accurately position Bph14.
^{b}From this table we can find that the molecular marker 76-2 cosegregates with resistance phenotype. The result shows that Bph14 locates between molecular marker SM1 and G1318 and cosegregates with 76-2.

One embodiment of the present invention provides an isolated nucleic acid molecule comprising a nucleotide sequence that comprises a brown planthopper resistance gene Bph14 selected from the group consisting of SEQ ID NO: 1 and SEQ ID NO: 2. In another embodiment, the nucleotide sequence encodes a polypeptide molecule comprising the amino acid sequence SEQ ID NO: 3. In yet another embodiment, the nucleotide sequence is operably linked to a heterologous promoter.

Another embodiment of the present invention provides an expression vector comprising the isolated nucleic acid molecule comprising a nucleotide sequence that comprises a brown planthopper resistance gene Bph14 selected from the group consisting of SEQ ID NO: 1 and SEQ ID NO: 2. In yet another embodiment, the present invention provides a transgenic plant, plant tissue, or plant cell comprising the expression vector. In still yet another embodiment, the transgenic plant, plant tissue, or plant cell is a monocot. In further yet another embodiment, the transgenic plant, plant tissue, or plant cell is rice.

Yet another embodiment of the present invention provides a method for producing a transgenic plant which expresses a Bph14 gene, comprising the steps of: (a) stably transforming a cell of a plant with a nucleic acid molecule comprising a nucleic acid sequence selected from the group consisting of SEQ ID NO: 1 and SEQ ID NO: 2 to produce a transformed cell; (b) regenerating a transgenic plant from the transformed cell; and (c) growing the transgenic plant wherein the nucleic acid molecule is expressed. In another embodiment, the transgenic plant is a monocot. In still yet another embodiment, the transgenic plant is rice.

Further yet another embodiment of the present invention provides a molecular marker associated with brown planthopper resistance, wherein the molecular marker is selected from the group consisting of: SG1, SG6, SG9, RM570, SM1, 76-2, and SM4. In one embodiment, SG1 is amplified by primers SEQ ID NOs: 14 and 15. In another embodiment, SG6 is amplified by primers SEQ ID NOs: 16 and 17. In yet another embodiment, SG9 is amplified by primers SEQ ID NOs: 18 and 19. In still yet another embodiment, RM570 is amplified by primers SEQ ID NOs: 20 and 21. In further yet another embodiment, SM1 is amplified by primers SEQ ID NOs: 22 and 23. In another embodiment, 76-2 is amplified by primers SEQ ID NOs: 24 and 25. In yet another embodiment, SM4 is amplified by primers SEQ ID NOs: 26 and 27.

Still yet another embodiment of the present invention is a method for determining the presence or absence of brown planthopper resistance in a plant or seed, comprising analyzing genomic DNA from the plant or seed for the presence of a molecular marker linked to a quantitative trait locus associated brown planthopper resistance, wherein the molecular marker is selected from the group consisting of: SG1, SG6, SG9, RM570, SM1, 76-2, and SM4. In another embodiment, the method further comprises analyzing genomic DNA from a plant or seed for the presence of a second molecular marker linked to a quantitative trait locus associated with brown planthopper resistance, wherein the second molecular marker is G1318. In yet another embodiment, the plant or seed is a monocot. In still yet another embodiment, the plant or seed is rice.

Another embodiment of the present invention is a quantitative trait locus associated with brown planthopper resistance, wherein the quantitative trait locus is located in a 34 kb region between a first molecular marker and a second molecular marker on chromosome 3 of rice. In another embodiment, the quantitative trait locus comprises Bph14.

1. A molecular marker associated with brown planthopper resistance, wherein the molecular marker is selected from the group consisting of: SG1, SG6, SG9, RM570, SM1, 76-2, and SM4.

2. The molecular marker of claim 1, wherein SG1 is amplified by primers SEQ ID NOs: 14 and 15.

3. The molecular marker of claim 1, wherein SG6 is amplified by primers SEQ ID NOs: 16 and 17.

4. The molecular marker of claim 1, wherein SG9 is amplified by primers SEQ ID NOs: 18 and 19.

5. The molecular marker of claim 1, wherein RM570 is amplified by primers SEQ ID NOs: 20 and 21.

6. The molecular marker of claim 1, wherein SM1 is amplified by primers SEQ ID NOs: 22 and 23.

7. The molecular marker of claim 1, wherein 76-2 is amplified by primers SEQ ID NOs: 24 and 25.

8. The molecular marker of claim 1, wherein SM4 is amplified by primers SEQ ID NOs: 26 and 27.

9. A method for determining the presence or absence of brown planthopper resistance in a plant or seed, comprising analyzing genomic DNA from the plant or seed for the presence of a molecular marker linked to a quantitative trait locus associated brown planthopper resistance, wherein the molecular marker is selected from the group consisting of: SG1, SG6, SG9, RM570, SM1, 76-2, and SM4.

10. The method of claim 9, further comprising analyzing genomic DNA from a plant or seed for the presence of a second molecular marker linked to a quantitative trait locus associated with brown planthopper resistance, wherein the second molecular marker is G1318.

11. The method of claim 9, wherein the plant or seed is a monocot.

12. The method of claim 11, wherein the plant or seed is rice.

13. A quantitative trait locus associated with brown planthopper resistance, wherein the quantitative trait locus is located in a 34 kb region between a first molecular marker and a second molecular marker on chromosome 3 of rice.

14. The quantitative trait locus of claim 13, wherein the quantitative trait locus comprises Bph14.
